(19) United States
(12) Patent Application Publication
(10) Pub. No.: US 2024/0156798 A1
(43) Pub. Date: May 16, 2024

(54) ANTIOXIDANT COMPOSITIONS AND METHODS OF USE

(71) Applicant: Sanare Pharmaceuticals, Del Mar, CA (US)

(72) Inventor: Dennis J. Carlo, Rancho Santa Fe, CA (US)

(21) Appl. No.: 18/493,435

(22) Filed: Oct. 24, 2023

Related U.S. Application Data
(60) Provisional application No. 63/420,221, filed on Oct. 28, 2022.
(60) Provisional application No. 63/429,671, filed on Dec. 2, 2022.

(51) Int. Cl. A61K 31/445; A61K 31/198; A61K 31/575; A61P 27/06; A61P 35/00

(52) U.S. Cl. CPC A61K 31/445; A61K 31/198; A61K 31/575; A61P 27/06; A61P 35/00

(57) ABSTRACT

Disclosed herein are compositions comprising antioxidants and methods of use thereof.

### CROSS-REFERENCE TO RELATED APPLICATIONS

The present application claims priority to U.S. Provisional Patent Application No. 63/420,221 filed Oct. 28, 2022, and U.S. Provisional Patent Application No. 63/429,671, filed Dec. 2, 2022, the entire contents of each of which are incorporated by reference herein in their entirety.

### FIELD

The present Specification relates to the use of antioxidants to treat various conditions, for example conditions related to inflammation.

### BACKGROUND

The main force behind acute inflammation is the body's immune system; when the body detects damaged tissue, cells called phagocytes rush to the site, ingesting disease-causing microorganisms or damaged cells as well as other foreign materials. This process is usually accompanied by the four “cardinal” signs of inflammation first noted by the Greek physician Celsus in the 2nd century C.E.; redness, heat, swelling and pain.

In injured tissues, blood vessels widen and blood flow surges, causing redness and heat. The walls of inflamed vessels become more porous, allowing inflammatory cells, proteins and fluids to leak into tissues, creating swelling and pressuring nerve endings.

With many infections, the disease-causing microorganism itself or a measured immune reaction to it triggers most symptoms, for example fevers which alert the body to an attack, or the coughs and loose stools that expel microscopic infectious particles. But if the immune system doesn't succeed in managing the organism or other source of cell trauma, it can resort to a “flood” of inflammation, wherein immune cells produce high volumes of cytokines; small proteins that act as messengers in an attempt to fight the infection.

Many people in intensive care units succumb to this burst of inflammation rather than to the infection itself; an overzealous immune reaction helps to explain why some young and ostensibly healthy individuals suffer severe illness or death during pandemics like SARS CoV 2.

Inflammation can persist with or without a known trigger, destroying healthy tissue. Autoimmune diseases such as arthritis or lupus, which turn inflammation against the body, can be devastating and sometimes fatal. Patients can suffer from inflammatory bowel disease (IBD), an autoimmune condition in which severe intestinal inflammation can require surgical intervention to remove most or all of the intestines. Additionally, many others cope with inflammation from acid reflux, food sensitivities, celiac disease, irritable bowel syndrome (IBS), and more.

Antioxidants are known to provide therapeutic benefits in a number of treatment scenarios, such as inflammation-associated conditions. However, thus far their potential has not been realized in a number of areas. For example, Crohn's Disease (CD) is a type of IBD causing swelling of the tissues (inflammation) in the digestive tract, which can lead to abdominal pain, severe diarrhea, fatigue, weight loss and malnutrition. “Fatty” liver and macular degeneration are also associated with inflammation.

Further, it is known that chemotherapy destroys both cancer cells and healthy cells. Conditions such as sinusoidal obstruction syndrome (SOS), steatosis, pseudocirrhosis and even hepatic necrosis can occur as an inflammation-related result of chemotherapy. Because medications are mainly broken down in the liver, liver damage can be a major side effect of chemotherapy. Many drugs require adequate liver function to be metabolized, and the same drugs can induce significant liver injury. In some cases, patients can be managed with supportive therapies and therefore liver toxicity may resolve after discontinuation of the chemotherapy.

Chemotherapy-induced liver toxicity can present in a multitude of forms. Free radicals that result from hepatocellular transformation may impair liver function. Also, when systemic doses of otherwise non-hepatotoxic chemotherapeutic agents are combined with low doses of radiation therapy, hepatic injury can result. Even treatment with agents like cytokines (for example IL-2) can result in a cholestatic pattern that can elevate many liver enzymes. This issue is yet to be adequately addressed.

Therefore, improved compositions and methods for treating inflammation-related conditions are desirable.

### SUMMARY

The instant disclosure provides compositions comprising antioxidants and methods of their use in a number of fields, such as treatment and prevention of inflammation-associated conditions. Disclosed compositions can comprise multiple antioxidants and demonstrate synergistic effects.

For example, disclosed compositions can comprise at least one antioxidant, such as tauroursodeoxycholic acid (TUDCA):

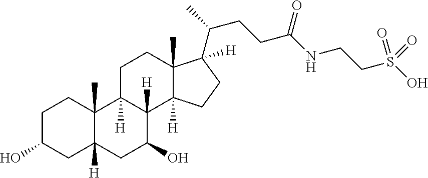

Disclosed compositions can comprise N-acetyl-cysteine (NAC:C₅H₉NO₃S):

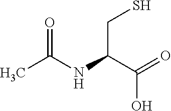

NAC is a drug approved by the Food and Drug Administration and used widely for the treatment of acetaminophen overdose (paracetamol). It is also approved for use in conditions with abnormal viscid or thickened mucous secretions such as pneumonia, bronchitis, tracheobronchitis, cystic fibrosis, post-traumatic chest conditions and before diagnostic bronchoscopy to help with mucus plugging. Off-label indications include acute hepatic failure, prevention of contrast-induced nephropathy and topical treatment of keratoconjunctivitis sicca.

Disclosed compositions can comprise 4-Hydroxy-TEMPO or TEMPOL, formally 4-hydroxy-2,2,6,6-tetramethylpiperidin-1-oxyl:

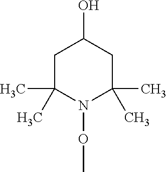

Disclosed compositions can comprise at least one of TUDCA, NAC, and TEMPOL.

Disclosed compositions can comprise at least two of TUDCA, NAC, and TEMPOL.

Disclosed compositions can comprise TUDCA, NAC, and TEMPOL.

Disclosed compositions can consist of at least one of TUDCA, NAC, and TEMPOL.

Disclosed compositions can consist of at least two of TUDCA, NAC, and TEMPOL.

Disclosed compositions can consist of TUDCA, NAC, and TEMPOL.

Disclosed compositions can comprise a pharmaceutically-acceptable carrier.

Disclosed compositions can comprise any pharmaceutically-acceptable form, such as solid dosage forms including pills and tablets. In embodiments, the solid dosage form can dissolve at or above a particular pH value. For example, in embodiments comprising treatment of CD the solid dosage form can be formulated to dissolve at or above, for example, 7.0. By providing compositions that dissolve at specific pH values, the compositions can be more accurately targeted, for example to tissues and organs beyond the stomach.

Disclosed compositions can comprise liquid solutions, for example for use topically or via injection, for example intravenous injection.

Disclosed compositions can be administered via any appropriate route, such as topical, oral, or parenteral administration. In some embodiments, TEMPOL/TUDCA/NAC and/or combinations thereof are administered topically (cutaneous and/or transdermal), orally, sublingually, transrectally, by ocular administration, intramuscularly, subcutaneously, or intravenously.

The compositions disclosed herein may be administered individually, i.e. sequentially in any order, or simultaneously. Simultaneous administration may be via any acceptable dosage form and route of administration. Sequential administration may be performed using individually packaged preparations. In one embodiment oral dosage forms may be provided to the patent is a blister back having multiple individual composition in the same package.

Disclosed methods comprise methods for treating a number of inflammation-associated conditions such as, but not limited to, IBDs (including CD and ulcerative colitis (UC)), Nonalcoholic steatohepatitis (NASH), Fatty Liver Disease (FLD), macular degeneration, and glaucoma. Further embodiments comprise limiting or preventing chemotherapy-induced liver toxicity. In embodiments, disclosed methods can comprise administration to a patient prior to, concurrently with, or following chemotherapy.

Disclosed methods can comprise administration of compositions comprising combinations of antioxidants, or administration of multiple compositions, each comprising a single antioxidant. Disclosed methods can comprise administration of compositions in different forms. For example, in embodiments, a liquid antioxidant composition can be administered with a solid composition, or a capsule can be administered with a powder or spray, etc.

### BRIEF DESCRIPTION OF THE FIGURES

FIG. 1 shows the physical appearance of a “fatty” liver.

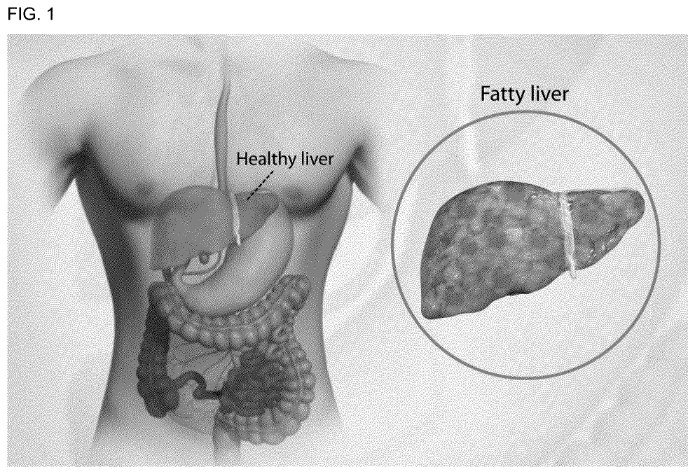

FIG. 2 shows the back of the eye with macular degeneration visible.

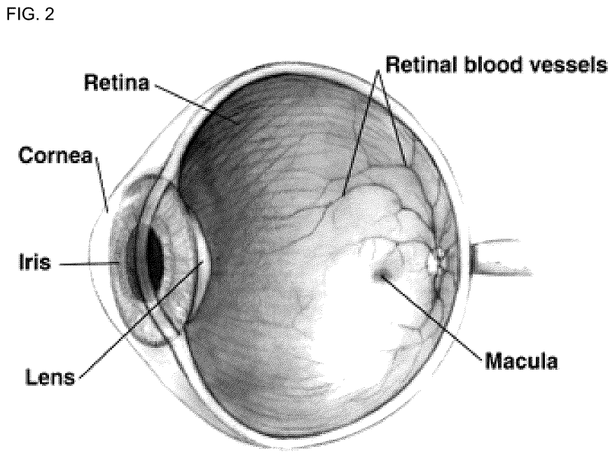

FIG. 3 shows an Amsler grid test which can help identify the distorted vision typical of macular degeneration.

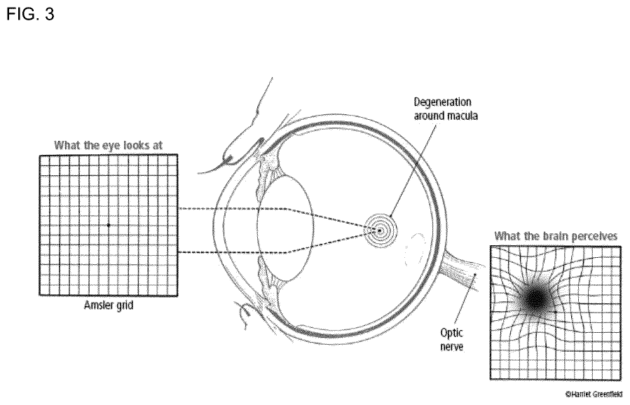

### DETAILED DESCRIPTION

TUDCA and UDCA are pleiotropic agents with multiple cellular targets that can inhibit apoptosis and up-regulate survival pathways. It is known that unfolded protein response (UPR), endoplasmic reticulum stress, and apoptosis are all important factors involved in many diseases, and that the inhibition of these events can change the course or onset of disease. It is not a surprise that the Chinese have used bear bile to treat a multitude of diseases for many hundreds of years; TUDCA and UDCA make up more than 50% of the “bile pool” in bear bile compared to just 2% in humans.

### Definitions

“Administration,” or “to administer” means the step of giving (i.e. administering) a medical device, material or agent to a subject. The materials disclosed herein can be administered via a number of appropriate routes.

“Associated with chemotherapy” means the condition (such as toxicity) arises as a result of the chemotherapy.

“Patient” means a human or non-human subject receiving medical or veterinary care.

“Parenteral administration” and “administered parenterally” are art-recognized terms, and include modes of administration other than enteral and topical administration, such as injections, and include, without limitation, retro-orbital, intraocular, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

“Pharmaceutically acceptable” or “therapeutically acceptable” refers to a substance which does not interfere with the effectiveness or the biological activity of the active ingredients and which is not toxic to a patient

“Pharmaceutically acceptable carrier” is art-recognized, and includes, for example, pharmaceutically acceptable materials, compositions or vehicles, such as a liquid or solid filler, diluent, excipient, solvent, or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Exemplary materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, sunflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

“Pharmaceutical composition” means a formulation including an active ingredient. The word “formulation” means that there is at least one additional ingredient (such as, for example and not limited to, an albumin [such as a human serum albumin or a recombinant human albumin] and/or sodium chloride) in the pharmaceutical composition in addition to an active ingredient. A pharmaceutical composition is therefore a formulation which is suitable for diagnostic, therapeutic or cosmetic administration to a subject, such as a human patient. The pharmaceutical composition can be: in a lyophilized or vacuum dried condition, a solution formed after reconstitution of the lyophilized or vacuum dried pharmaceutical composition with saline or water, for example, or; as a solution that does not require reconstitution. As stated, a pharmaceutical composition can be liquid, semi-solid, or solid. A pharmaceutical composition can be animal-protein free. A pharmaceutical composition disclosed herein can optionally include, without limitation, other pharmaceutically acceptable components (or pharmaceutical components) including, without limitation, buffers, preservatives, tonicity adjusters, salts, osmolality adjusting agents, physiological substances, pharmacological substances, bulking agents, emulsifying agents, wetting agents, flavoring agents, coloring agents, suspension agents, and the like.

“Reducing,” “suppressing” and “inhibiting” have their commonly understood meaning of lessening or decreasing.

“Therapeutic formulation” means a formulation that can be used to treat and thereby alleviate a disorder or a disease and/or symptom associated thereof.

“Therapeutically effective amount” means the level, amount or concentration of an agent, material, or composition needed to achieve a treatment goal.

“Treat,” “treating,” or “treatment” means an alleviation or a reduction (which includes some reduction, a significant reduction, a near total reduction, and a total reduction), resolution or prevention (temporarily or permanently) of a symptom, disease, disorder or condition, so as to achieve a desired therapeutic or cosmetic result, such as by healing of injured or damaged tissue, or by altering, changing, enhancing, improving, ameliorating and/or beautifying an existing or perceived disease, disorder or condition.

Antioxidant Compositions

Disclosed embodiments comprise pharmaceutical compositions. In embodiments, the compositions can comprise any appropriate form, for example liquids such as aqueous liquids, solids, semi-solids, gels, extended-release forms such as implants, and the like.

Disclosed compositions can comprise at least one antioxidant; compounds that inhibit oxidation, a chemical reaction that can produce free radicals and chain reactions that may damage cells. For example, disclosed compositions can comprise antioxidants of a number of classes. For example, antioxidants suitable for use with disclosed embodiments can be classified as enzymatic or non-enzymatic antioxidants:
a. Dangerous oxidative products can be converted to H₂O₂ and then to water by enzymatic antioxidants that are able to break down free radicals in a multistep process in the presence of cofactors such as copper (Cu), zinc (Zn), manganese (Mn), selenium (Se), and iron (Fe).
b. In contrast, vitamin C, vitamin E, plant polyphenol, carotenoids, and glutathione are non-enzymatic antioxidants, which act by interrupting free radical chain reactions.
c. According to size, antioxidants can be categorized as small or large-molecule antioxidants. The small molecule antioxidants neutralize the reactive oxygen species (ROS) in a process named radicals scavenging. Vitamin C, vitamin E, carotenoids, and glutathione (GSH) are the main antioxidants in this category.
d. Large molecule antioxidants include enzymes (SOD, CAT, and GPx) and sacrificial proteins (albumin) that absorb ROS and prevent them from attacking other essential proteins.

Disclosed compositions can comprise any of enzymatic, non-enzymatic antioxidants, small molecule, or large molecule antioxidants. For example, disclosed compositions can comprise TUDCA, a non-enzymatic antioxidant:

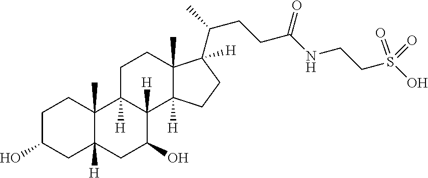

TUDCA is the taurine conjugate of ursodeoxycholic acid (UDCA) and is Food and Drug Administration (FDA) approved for primary biliary cholangitis. UDCA has been approved for the treatment of certain cholestatic liver diseases due to its ability to protect hepatocytes. TUDCA and UDCA have been shown to act as potent inhibitors of apoptosis, acting by interfering with the mitochondrial pathway of cell death. In addition, they have been shown to inhibit ROS, reduce endoplasmic reticulum (ER) stress, and stabilize the unfolded protein response (UPR).

TUDCA has been shown to provide protection and down-regulate the inflammation cascade:
a. inhibits epithelial cytokine release;
b. inhibits immune cell recruitment;
c. inhibits immune cell activation;
d. inhibits apoptosis (cell death) caused by inflammation and impaired barrier function;
e. promotes restitution of epithelial cells;
f. restores the mucus layer;
g. restores microbiota;
h. inhibits ROS production;
i. prevent Bax-induced mitochondrial membrane perturbation;
j. downregulates the unfolded protein response (UPR);
k. block endoplasmic reticulum (ER) stress;
l. modulates cell cycle protein expression;
m. activates bile acid receptor famesoid X.

Similarly, N-acetyl-L-Cystein (NAC) is a non-enzymatic antioxidant that can be employed in disclosed compositions:

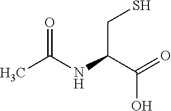

NAC is approved by the FDA and used widely for the treatment of acetaminophen overdose (paracetamol). It is also approved for use in conditions with abnormal viscid or inspissated mucous secretions such as pneumonia, bronchitis, tracheobronchitis, cystic fibrosis, post-traumatic chest conditions and before diagnostic bronchoscopy to help with mucus plugging. Off-label indications include acute hepatic failure, prevention of contrast-induced nephropathy and topical treatment of keratoconjunctivitis sicca. There are even animal and human studies showing its use in decreasing cisplatin-induced nephrotoxicity.

NAC has been shown to protect in an acetic acid-induced experimental colitis model in rats. Colitis caused by acetic acid has been characterized by an increase in myeloperoxidase (MPO) activity, as indicator of accumulation or infiltration into the colon by polymorphonuclear neutrophils. Treatment with NAC (100 mg/kg) for seven days, compared to vehicle controls, showed a significant decrease in colon tissue MPO activity and a restoration of GSH and NO concentrations. Macroscopic and histological findings also suggested the protective role of NAC; several effects of NAC are listed in the Table below:

| Action | Mechanism |
|---|---|
| Action on Glutathione | NAC restores glutathione (cysteine is rate limiting) |
| Stabilizes proteins/DNA | Protects proteins by crosslinking cysteine disulfide |
| | molecules. Various mechanisms of DNA |
| | repair/protection |
| Scavenges free radicals | Scavenging property via the redox potential of |
| | thiols |
| Anti-inflammatory property | Reduces pro-inflammatory cytokines |
| Antioxidant property | Reduces oxidative damage, elimination of reactive |
| | species |
| Mucolytic property | Splits disulfide bonds in mucoproteins lowering |
| | viscosity |
| Mitochondrial resilience | Neurogenesis inducing ability and reduces |
| | apoptosis of mitochondria |
| Metal chelation | Thiol groups provide binding sites for metals |
| Glutamate/dopamine | Modulates glutamate and dopamine |
| homeostasis | |
| Action on Glutathione | NAC restores glutathione (cysteine is rate limiting) |
| Antiviral properties | Immune modulation, anti-NFκB properties, and |
| | other unexplored mechanisms observed in vitro |
| | and in vivo |
| Vascular endothelial growth | Inhibition of vascular permeability as seen in |
| factor | human keratinocytes |
| Adenosine triphosphate (ATP) | Increased ATP production in some cells like |
| and nitric oxide (NO) production | fibroblasts. Increased nitric oxide production as |
| | demonstrated in human studies |

Similarly, 4-Hydroxy-TEMPO or TEMPOL, formally 4-hydroxy-2,2,6,6-tetramethylpiperidin-1-oxyl is a non-enzymatic antioxidant that can be employed in disclosed compositions:

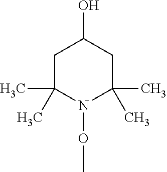

TEMPOL is a synthetic antioxidant with an excellent profile in that it is safe and well tolerated for acute or chronic dosing. Its half-life is consistent with dosing once or twice daily. Efficacy has been observed in many reported studies and over 2,000 articles have been published, including many from the National Cancer Institute and the FDA. It can be administered as an oral liquid, solid, topical or intravenously. Additionally, it prevents and reverses the production of free radicals thereby preventing or reducing oxidative stress created by such things as infection, inflammation, tissue injury, chemotherapy and radiation.

TEMPOL favors the metabolism of a wide variety of cellular reactive oxygen and nitrogen species and reduces oxidative stress. TEMPOL is uncharged and transports through membranes into cells. It is non-toxic and non-immunogenic for normal cells. It does not react with biological molecules nor dimerize. Structural steric hindrance only allows reactions with small reactive oxygen species. More specifically, TEMPOL:
a. is a redox-cycling nitroxide that promotes the metabolism of many reactive oxygen species (ROS) and improves nitric oxide bioavailability;
b. neutralizes reactive oxygen species (ROS) generated by infections, tissue injury, inflammation, radiation and/or chemotherapy and acts as a SOD mimetic;
c. acts as both an anti-oxidant and anti-inflammatory;
d. reduces oxidative stress by repairing damaged mitochondria to prevent increased free radical production. It restores mitochondrial stability and viability;
e. has been shown to downregulate inflammatory cytokines IL-6, IL-8, IL-1 beta, TNF-alpha, interferon-gamma, etc.;
f. blocks NF-κB, a key regulator of inflammation; and
g. decreases production of cytokines in the retina and optic nerve. Gene regulation effects:

Gene regulation research studies on TEMPOL have shown that it has the ability to regulate a variety of genes, e.g.;
a. statistically increased expression of the ADIPOQ gene. Increasing ADIPOQ reduces the inflammatory responses;
b. It is also a crucial regulator of pro-inflammatory TNFα, in that it downregulates TNFα;
c. positively impacts genes that regulate cell cycle and programmed cell death (apoptosis). TEMPOL significantly increases UNC5b to promote cell death in cancer cells;
d. significantly increased Glutathione S-Transferase (GST) gene(s) expression. GST genes are essential for cellular defense in response to devastating free radicals;
e. statistically increased expression levels of keratinocyte growth factor (KGF) gene. KGF stimulates the proliferation and differentiation of epithelial cells;
f. significantly decreased expression of genes encoding for the 26s proteasome complex. Abnormal cells naturally increase expression of these genes which lead to the increase of misfolded proteins;
g. statistically decreased expression of HIF-1α and HIF-2α. Elevated levels strongly correlate with metastasis, tumor resistance, and ocular disease progression.

The beneficial biological effects of TEMPOL range from protective effects against cancer, radiation, chemotherapy, metabolic syndrome and shock as well as inflammatory bowel disorders, eye, liver, heart, kidney and central nervous system safe-keeping. In addition, TEMPOL has been shown to decrease platelet activation, decrease pulmonary inflammation, protect non-cancerous cells from various toxic agents including chemotherapeutic agents such as paclitaxel, doxorubicin and cisplatin, and alleviate and prevent chemotherapy-induced neuropathic pain by reducing the levels of inflammatory cytokines and free radicals in dorsal root ganglia.

TEMPOL has been studied in hundreds of animal models of oxidative stress and inflammation. No safety concerns have been observed. TEMPOL as a topical application has been shown to decrease ocular damage in rat, and rhesus monkey models.

Because of its broad activity, TEMPOL has the potential to be more efficacious than a single anti-cytokine agent. TEMPOL has been used topically in multiple human studies of radiation dermatitis and has been shown to protect against radiation induced alopecia in human clinical trials. These alopecia studies were conducted by the former head of the FDA. TEMPOL (oral formulation) is currently being tested in a Phase 2 trial of radiation induced mucositis in head and neck cancer.

Disclosed compositions can comprise at least one of TUDCA, NAC, and TEMPOL.

Disclosed compositions can comprise at least two of TUDCA, NAC, and TEMPOL.

Disclosed compositions can comprise TUDCA, NAC, and TEMPOL.

Disclosed compositions can consist of at least one of TUDCA, NAC, and TEMPOL.

Disclosed compositions can consist of at least two of TUDCA, NAC, and TEMPOL.

Disclosed compositions can consist of TUDCA, NAC, and TEMPOL.

Methods of Treatment

Disclosed embodiments comprise methods of treatment, for example methods comprising administration of at least one composition described herein. Disclosed methods can comprise treatment of, for example, the following:

Inflammatory Bowel Diseases

IBD has been a global healthcare problem with a sustained increasing incidence. IBD is a chronic inflammation of the gastrointestinal tract (GI). IBDs are multifactorial disorders characterized by genetic susceptibility, immune cell overactivation, microbial gut dysbiosis and changes in intestinal barrier permeability. IBDs normally further classified as CD or UC which are differentiated by their location and by depth of involvement in the bowel wall. Both inflammatory conditions increase the risk of colon cancer. In embodiments, the combination of TUDCA and NAC can provide synergistic effects in the treatment of IBD:

| TUDCA | NAC |
|---|---|
| Inhibits reactive oxygen species (ROS) | Acts as an antioxidant and anti- |
| and is anti-inflammatory | inflammatory; scavenging property via |
| | the redox potential of thiols |
| Potent inhibitor of apoptosis | Inhibits apoptosis; reduced expression |
| | of the caspase-3 protein (apoptosis) in |
| | colonic mucosa |
| Interferes with mitochondrial pathway of | Reduces mitochondrial apoptosis |
| cell death | |
| Reduces endoplasmic reticulum (ER) | Reduces ER stress |
| stress (prevents Grp78 upregulation) | |
| Modulator of intestinal permeability | Contributes to the tightening of the |
| | intestinal barrier |
| Activates the intestinal farnesoid X | Prevents activity of NFκB which is |
| receptor (FXR); anti-inflammatory effects | involved in the activation of pro- |
| of FXR signaling | inflammatory cytokines |
| Activates TGR5 and leads to decrease in | Decreases pro-inflammatory cytokine |
| IL-6, IFNγ and TNFα | production |
| Stabilizes unfolded protein | Protects proteins by crosslinking |
| | cysteine disulphide molecules. Affects |
| | cytoskeleton structure |
| Inhibits immune activation and | Immuno-modulating activity and blocks |
| recruitment | NFκB |
| Activation of bile acid receptors in | Inhibits pro-inflammatory cytokines and |
| immune cells; modulates immune | has immuno-modulating activity |
| response and alters inflammatory | |
| reactions | |
| Inhibits pro-inflammatory cytokines | Downregulates pro-inflammatory |
| | cytokines |
| Improves intestinal barrier function and | Tightens intestinal barrier by influencing |
| intestinal permeability | proteins of the claudin and occludin |
| | groups |
| Modulates intestinal microbiota | Positive effect on intestinal flora |
| composition | |
| Increases Vit. D Receptor (VDR); results | Both antioxidant and anti-inflammatory |
| in anti-inflammatory effects | effects |
| Decreases neutrophil infiltration and | Decreases inflammatory neutrophil cell |
| activity as well as a general decrease in | infiltration |
| leukocyte infiltration | |
| Active against chemically induced colitis | Protects against chemically induced |
| | colitis |
| Decreases intestinal inflammation | Protects against intestinal inflammation |
| Beneficial effect on systemic and | Has effects on both systemic and |
| intestinal immunity | intestinal immunity |
| Positive results in an open label Phase 1 | Positive results in a Phase 2 (168 |
| study testing efficacy and safety in U.C. | patients) double blind, placebo- |
| patients | controlled study |
| | Contributes to levels of mitochondrial |
| | ATP and increases blood flow |
| | (vasodilator) |
| Promotes restitution of epithelial cells | Promotes the process of proliferation |
| | and regeneration of intestinal cells |

Crohn's Disease/Ulcerative Colitis

CD is chronic inflammation in the gastrointestinal tract that spreads through the bowel wall. It may involve any part of the digestive tract although the small intestine, particularly the ileum, is the most commonly involved organ with the colon the next most commonly involved organ. Crohn's disease is an autoimmune disease. With CD, once the inflammatory response is triggered by the immune system, it doesn't subside. Regarding CD treatments, decreased blood and mucosal levels of antioxidant vitamins A, C, E and β-carotene have been reported in Crohn's patients. It has been shown that antioxidants inhibit the production of inflammatory cytokines, such as IL-1 and TNFα, in the colonic mucosa of inflammatory bowel disease patients including those with CD. Further, TUDCA decreases inflammatory cytokines, including TNF-alpha, induces resolution of ER stress in intestinal epithelial cells and improves the function of the tight junction, lipid transport and gut microbiota.

UC is a type of inflammatory bowel disease along with Crohn's disease and microscopic colitis. It is a long-term condition that results in inflammation and ulcers of the colon and rectum. The primary symptoms of active disease are abdominal pain and diarrhea mixed with blood. Weight loss, fever, and anemia may also occur. Often, symptoms come on slowly and can range from mild to severe. Symptoms typically occur intermittently with periods of no symptoms between flares. Complications may include abnormal dilation of the colon, inflammation of the eye, joints, or liver, and colon cancer.

Combinations as disclosed herein can provide a synergistic treatment effect by combining the following effects, including synergistic effects, for example in the treatment of UC and CD:

| TUDCA | TEMPOL |
|---|---|
| Inhibits reactive oxygen species (ROS) | Prevents and reverses the production of |
| and is anti-inflammatory | ROS and acts as a SOD mimetic |
| | Promotes the metabolism of many ROS |
| | and improves nitric oxide bioavailability |
| | and is anti-inflammatory |
| Potent inhibitor of apoptosis | Positively impacts genes that regulate cell |
| | cycle and programmed cell death |
| | (apoptosis) |
| Interferes with mitochondrial pathway of | Restores mitochondrial stability and |
| cell death | viability |
| Reduces endoplasmic reticulum stress | |
| (prevents Grp78 upregulation) | |
| Modulators of intestinal permeability | |
| Activates the intestinal farnesoid X | Acts as an anti-inflammatory and |
| receptor (FXR); anti-inflammatory effects | antioxidant |
| of FXR signaling | |
| Activates TGR5 and leads to decrease in | Decreases pro-inflammatory cytokines |
| IL-6, IFNγ and TNFβ | |
| Stabilizes unfolded protein | Significantly decreases expression of |
| | genes encoding the 26s proteasome |
| | complex, which reduces the expression of |
| | misfolded proteins |
| Inhibits immune activation and recruitment | Blocks NFκB, a key regulator of |
| | inflammation |
| Activation of bile acid receptors in immune | Decreases production of cytokines and |
| cells; modulates the immune response | inflammatory response |
| and alters inflammatory reactions | |
| Inhibits pro-inflammatory cytokines | Downregulates inflammatory cytokines: |
| | IL-6, IL- 8, IL-1beta, TNFα, Interferonγ |
| | etc. |
| Promotes restitution of epithelial cells | Stimulates proliferation and differentiation |
| | of epithelial cells |
| Improves intestinal barrier function and | |
| intestinal permeability | |
| Modulates intestinal microbiota | |
| composition | |
| Increases Vitamin D Receptor (VDR); | Antioxidant and anti-inflammatory |
| results in anti-inflammatory effects | |
| Decreases neutrophil infiltration and | Reduction in the degree of both neutrophil |
| activity as well as general decrease in | inflammation and lipid peroxidation in the |
| leukocyte infiltration | inflamed bowel |
| Active against chemically induced colitis | Reduction in the degree of both neutrophil |
| | inflammation and lipid peroxidation in the |
| | inflamed bowel |
| Decreases in intestinal inflammation | Effectively decreases mucosal damage in |
| | experimental colitis |
| Beneficial effect on systemic and intestinal | Increases expression of Glutathione S- |
| immunity | Transferase (GST) gene(s), which are |
| | essential for cellular defense in response |
| | to devastating free radicals |
| Prevents Bax induced mitochondrial | Restores mitochondrial stability and vitality |
| membrane perturbation | |
| Positive results in Phase 1 study testing | |
| efficacy and safety in U.C. Patients | |

Glaucoma/Macular Degeneration

Glaucoma is a group of eye diseases that result in damage to the optic nerve (or retina) and cause vision loss. The most common type is open-angle (wide angle, chronic simple) glaucoma, in which the drainage angle for fluid within the eye remains open, with less common types including closed-angle (narrow angle, acute congestive) glaucoma and normal-tension glaucoma. Open-angle glaucoma develops slowly over time and there is no pain. Peripheral vision may begin to decrease, followed by central vision, resulting in blindness if not treated.

Glaucoma is characterized by damage of the optic nerve and progressive degeneration of retinal ganglion cells (RGCs) which are critical elements for vision loss. Current glaucoma therapies target reduction in intraocular pressure (IOP), but since RGC death is the cause of irreversible vision loss, neuroprotection may be a good strategy for glaucoma treatment. Oxidative stress is an important risk factor in human glaucoma and consistently, the plasma levels of glutathione (GSH), an antioxidant, is decreased in glaucoma patients.

Oxidative stress induced signaling for neuroinflammation in glaucoma includes the stimulation of a transcriptional program for inflammatory mediators, such as IL-1, IL-2, IFN-α and TNFα. The ROS master transcriptional regulator of cytokine production, namely nuclear factor-kappa beta (NFκβ) is upregulated in human glaucoma and animal models. Results in C57BL/6J mice with experimentally induced glaucoma showed that tempol treatment decreased neuro-inflammation in the ocular hypertensive retina and optic nerve. It should be noted that TEMPOL is blood-brain barrier permeable and can readily access the intracellular compartment.

N-acetyl cysteine (NAC) has been shown to have a positive (protective) effect on primary cultures of human retinal pigment epithelium (RPE) from patients with AMD. NAC protects against oxidative stress by blocking excessive ROS accumulation and preventing both H₂O₂ induced cell death and GSH depletion in RPE from both positive and negative AMD donors. NAC also improved basal mitochondrial function (ATP) in both groups of cells. Strong experimental evidence supports the idea that mitochondrial damage is one of the key events driving AMD pathology.

Several beneficial effects specific for AMD RPE include reduction in basal ROS, an increase in basal GSH content and greater NAC protection after oxidation. It should be noted that RPE are adjacent to the choriocapillaris, the main source of oxygen for the outer retina, placing them in a highly oxidative environment. Also, the oxidizing environment within the RPE are the ROS generated as a product of the reaction of light with abundant photosensitizers (lipofuscin and melanin). The results support the relevance of NAC as a possible therapeutic for AMD taking into consideration the importance of maintaining RPE mitochondrial function for overall retinal health.

It should also be noted that in a mouse model of photo-induced retinal degeneration, intraperitoneal injection of NAC suppressed oxidation and ER stresses, while inhibiting ROS accumulation in the Balb/c mouse retina. Additionally, in a murine dry eye study, eye drop administration of NAC diminished the levels of ROS and inflammasome signaling. These studies also support the fact that NAC treatment reduces ROS and improves cell viability. In another mouse study, NAC was effective in protecting the retina from oxidative damage when applied topically to the eye. This was shown in rd 10+/+ mice, a model of retinitis pigmentosa. This has important implications in that when NAC is applied to the cornea, it is able to penetrate to the posterior segment and protect the retina.

NAC has a long history of successful use in multiple conditions where elevated ROS induces pathology. NAC has also been shown to have a protective effect on retinal degeneration in a mouse model of normal tension glaucoma (NTG). In EAAC1 KO mice (excitatory amino-acid carrier 1) oxidative stress and autophagy were suppressed with increased glutathione levels by NAC treatment. NAC prevented the decline of retinal function and was shown to be neuroprotective. NAC treatment also protected retinal ganglion cells (RGC) from NTG-like neurodegeneration. EAAC1 is expressed in retinal neurons including RGC. EAAC1 transports not only extracellular glutamate, but also cysteine, which is an important substrate for glutathione (GSH) synthesis, into neural cells. GSH has a strong protective role against oxidative stress as an antioxidant in the retina. Oxidative stress is one of the pathogenic factors for glaucoma. The plasma level of GSH is decreased in primary open angle glaucoma including NTG patients, supporting the fact that the mouse model is suitable for basic research of NTG. Therefore, targeting oxidative stress in the retina, combined with the treatment of IOP reduction, may be a therapeutic strategy to treat glaucoma.

Another bile acid receptor is transmembrane G-protein coupled receptor 5 (TGR5), which is widely distributed throughout the body. It has brought on new explorations of Bile Acid (BA)-based therapies and their extra-hepatic-homeostatic functions on lipid, glucose, and energy metabolism. This includes a number of ocular afflictions, including MD.

Macular degeneration damages the macula, which provides sharp, central vision. The macula is the most sensitive part of the retina. It is located at the back of the eye. The retina turns light into electrical signals and then sends them through the optic nerve to the brain, where they are translated into the images we see. There are two forms of macular degeneration:
a. Dry macular degeneration. Most people with macular degeneration have this type. The cells of the macula slowly break down. This produces blurring at first, then blank spots in the eye's central vision. In the beginning, the symptoms are subtle, then become more noticeable. Some cases of dry macular degeneration progress to the more serious wet macular degeneration.
b. Wet macular degeneration. Everyone with wet macular degeneration starts out with the dry form. At some point, new blood vessels begin to grow beneath the retina. The new vessels leak blood and fluid into the macula, causing scarring. Wet macular degeneration can cause rapid loss of vision over days to weeks and continued loss of vision over time.

TUDCA has recently been shown to block apoptosis following cellular injury in several models of neurodegeneration and damage, including models of retinal degeneration. The mechanism of action includes stabilizing the mitochondrial membrane, inhibiting apoptosis, and the prevention of endoplasmic reticulum (ER) stress.

Both mouse and rat optic crush models have been used to induce retinal ganglion cell (RCG) death. The loss of RGCs occurs in a way that is comparable to glaucoma (highly acute glaucoma model). TUDCA treatment has been used in these models with similar positive results. Systemic treatment with TUDCA significantly enhanced RCGs survival and suppressed apoptosis following optic nerve crush. In glaucoma, RCGs die in at least three ways: mechanical damage to the axon, vascular insufficiency and exocytotoxic damage. The surgical crush mimics the mechanical injury to the RCG axons exiting the eye and the indirect partial occlusion of the central retinal vein (which travels the optic nerve bundle) mimics glaucomatous vascular insufficiency. The exocytotoxic injury found in glaucoma is also mimicked in the optic nerve crush model, as the crush elevates both intraocular glutamate and aspartate levels.

Another model that causes corneal and retinal damage is the ocular alkali burn model (OAB). It is one of the most severe ophthalmic emergencies and is responsible for 6.9% to 13.2% of ocular injuries. In the OAB model, treatment with TUDCA was shown to inhibit ocular inflammation and protect the cornea and retina from injury. The healing and preservative action of TUDCA is even more effective in combination with TEMPOL.

Considering the mechanism of action and the current experimental evidence, the combination of TUDCA and NAC may very well play an important role in the treatment of glaucoma and MD; for example by combining the following effects, which can be synergistic:

| TUDCA | NAC |
|---|---|
| Decreases inflammation; protects cornea | Acts as an antioxidant and anti- |
| and retina from injury | inflammatory; scavenging property via the |
| | redox potential of thiols and protects |
| | cornea and retina |
| Inhibits ROS and inflammation | Inhibits ROS and suppresses NFκB |
| | activation and inflammation; reduction of |
| | basal ROS |
| Decreases inflammatory cytokines in the | Suppresses cytokine production (TNFα, |
| retina and optic nerve | IL-1 and IL-6) |
| Stabilizes mitochondrial membranes | Preserves mitochondrial function and |
| | prevents ROS production |
| Inhibits apoptosis and protects RGC | Inhibits cellular apoptosis/Caspase 3 |
| | expression and protects RGC |
| Protects against glial activation | Shown to be neuroprotective in protecting |
| | retinal ganglion cells |
| Suppresses angiogenesis | |
| Suppresses corneal neovascularization | Inhibition of vascular permeability |
| (CVN) and VEGF expression | |
| Strengthens the unfolded protein | Rescues the unfolded protein response |
| response | |
| Block ER stress; reduces expression of | Blocks ER stress |
| ER stress markers (IRE1, GRP78 and | |
| CHOP in retinal tissue | |
| Promotes corneal re-epithelization | Promotes wound healing |
| Preserves retinal structure | Protective effect on primary culture of |
| | human retinal pigment epithelium; |
| | promotes retinal health and targets |
| | oxidative stress in the retina |
| Inhibits NFκB | Blocks NFκB |
| Blood/brain barrier permeable | Applied to the cornea it penetrates to the |
| | posterior segment and protects the retina |
| Protects retinal and visual function in the | Prevents glutathione depletion and |
| mouse model | improves cell viability and prevents decline |
| | of retinal function (neuroprotective) |
| Slows retinal degeneration | Protective effect on retinal degeneration in |
| | the mouse model of normal tissue |
| | glaucoma (NTG) |
| Protects retinal ganglia cells in the optic | |
| crush model | |

Similarly, combination treatments of TUDCA and TEMPOL can be used to treat glaucoma and MD, and in cases provide synergistic effects:

| TUDCA | TEMPOL |
|---|---|
| Decreases inflammation; protects cornea | Decreases neuro-inflammation in retina |
| and retina from injury | and optic nerve |
| Inhibits ROS and inflammation | Neutralizes ROS |
| | Protects against hypoxia; decreases |
| | expression of HIF-1α and HIF-2α |
| Decreases inflammatory cytokines in the | Down-regulates inflammatory cytokines: |
| retina and optic nerve | IL-6, IL-8, IL-1, TNF, IFN, etc. |
| Stabilizes mitochondrial membrane | Repairs damaged mitochondria and |
| | promotes viability |
| Inhibits apoptosis and protects RGCs | Inhibits apoptosis and protects RGCs |
| Protects against glial activation | Protects against glial activation |
| Suppresses angiogenesis | |
| Suppresses corneal neovascularization | |
| (CVN) and VEGF expression | |
| Strengthens the unfolded protein | Protects against protein alteration |
| response | |
| Blocks ER stress. Reduces the | |
| expression of ER stress markers (IREI, | |
| GRP78 and CHOP) in the retinal tissues | |
| Promotes corneal re-epithelization | Increases expression of KGF gene, which |
| | stimulates proliferation and differentiation |
| | of epithelial cells |
| Preserves retinal structure | |
| | Increases expression of glutathione |
| | transferase (cellular defense in response |
| | to devastating free radicals) |
| Inhibits NFₖB | Blocks NFₖB, a key regulator of |
| | inflammation |
| Blood/brain barrier permeable | Blood/brain barrier permeable |
| Protects retinal ganglion cells in the optic | |
| crush model | |

Nonalcoholic Steatohepatitis (NASH)

The FDA has approved TUDCA for treatment of certain cholestatic liver diseases. TUDCA has been recently approved for Amyotrophic Lateral Sclerosis (ALS) (or Lou Gehrig's Disease) in Canada and in the U.S. TUDCA is also available over the counter as a supplement.

Numerous cancer drugs can cause blockage of the central and sublobular veins in the liver resulting in SOS and other liver problems, such as higher than normal liver enzymes and increased bilirubin levels. This can lead to toxicity and liver dysfunction. During chemotherapy, up to 85% of patients develop liver steatosis and this becomes more serious if accompanied by increased bilirubin levels. Together, all of this can be a sign of liver inflammation, which creates a significant risk of developing FLD and increases apoptosis.

Because the liver is the central organ of intermediate metabolism, metabolic detoxification and excretion of waste products, it becomes essential to have, as close to possible, normally functioning hepatocytes in order to properly administer chemotherapy.

To this end, TUDCA and UDCA have been shown to exert protective effects on the liver by decreasing apoptosis and interfering with the mitochondrial pathway of cell death, protecting liver enzymes and bilirubin and significantly inhibiting reactive oxygen species. Considering these attributes, TUDCA serves as an excellent candidate to protect the liver during chemotherapy treatment. In fact, TUDCA may permit the use of higher doses of chemotherapy, thereby leading to better clinical outcomes.

Liver anti-oxidant capacity is dependent on glutathione production to prevent free radical formation, and it is known that N-acetyl-L-Cystein (NAC) produces healthy glutathione levels, promoting normal liver function. Additionally, NAC is a powerful antioxidant that protects cells from oxidative stress. Because chemotherapy can have long term effects (cognition, hearing, heart, lung, blood, nerve and reproductive damage), dual therapy may allow the appropriate management to reduce morbidity and mortality. In addition to TUDCA, NAC has been shown to help with detoxification of kidney and liver damage, may improve cognitive health, may help relieve symptoms of respiratory conditions, may improve fertility in men and women and may stabilize blood sugar.

As to FLD, interest in bile acids (BA) arose from the discovery of their signaling properties and ability to regulate the activity of many genes through the orphan nuclear receptor (Famesoid X Receptor, RXR). FXR agonists have shown potential as treatment for primary biliary cirrhosis and nonalcoholic steatohepatitis.

The liver is the largest organ in the body. It helps the body digest food, store energy, and remove poisons. Fatty liver disease is a condition in which fat builds up in the liver. There are two main types differentiated by cause:
a. Alcoholic fatty liver disease, also called alcoholic steatohepatitis (AS)
b. Nonalcoholic fatty liver disease (NAFLD)

These two types can progress through subsequent stages of increasing severity to cirrhosis.

The gut-liver axis is associated with progression of NAFLD. Therefore, targeting the gut-liver axis with bile acid-based combination drug should prove beneficial in the treatment of NAFLD. Studies have shown that TUDCA fulfills the criteria. TUDCA treatment markedly reduces high fat diet induced NAFLD in mice. In addition, to its commonly accepted protective effect of ER stress, TUDCA attenuates gut inflammation, improves the intestinal barrier function, decreases intestinal fat transport, and modulates the intestinal microbiota composition.

There are indications that TUDCA could reduce the severity of NAFLD and NASH by reduction of liver inflammation. TUDCA would be used to protect the liver cells by down-regulating the inflammatory cascade, reducing the ROS, inhibiting apoptosis, reducing ER stress, and stabilizing the UPR.

TUDCA alone has been shown to attenuate progression of HFD induced NALFD in mice by ameliorating gut inflammation, improving intestinal barrier protection, decreasing intestinal fat transport and modulating intestinal flora. A combination drug to treat NASH will also play a significant role in reducing cardiovascular disease, chronic kidney disease and intra-hepatic and extra-hepatic malignancy, all of which have been associated with NAFLD.

The combination of TUDCA and NAC produces a broader spectrum of disease-fighting mechanisms than either one would have alone. This should enable the combination to have a more positive impact in treatment of complex diseases than any single drug. NASH is an example. Currently, there are no medications approved for NASH, though some are in clinical trials. The disease mechanisms that lead to NASH and fibrosis are complex, involving both metabolic and inflammatory pathways, including:
a. Insulin resistance and lipid metabolism;
b. Lipid toxicity and immune activation;
c. Cell death;
d. Fibrogenesis and collagen turnover.

The combination of TUDCA and NAC targets both metabolic dysfunction and cell damage. Based on published literature, TUDCA and NAC have complementary effects on many of the known biological mechanisms that are active in NASH.

Antioxidant activity plays a key role in protecting the liver, and NAC is a powerful anti-oxidant. NAC can confer benefits in disorders caused by oxidative stress (OS). For example, in mice fed high fat diets (HFD), NAC significantly reduced hepatic steatosis and metabolic disturbances as compared to normally fed mice. NAC totally restored normal morphology of hepatic tissue. Large lipid droplets and large adipocytes were shown to be higher in the HFD mice. Additionally, NAC treated mice showed improved glucose tolerance demonstrating that NAC could attenuate HFD-induced metabolic disturbance. Based on published literature, TUDCA and NAC have complementary effects on the known biological mechanisms of NASH. TUDCA alone has been shown to attenuate progression of HFD induced NALFD in mice by ameliorating gut inflammation, improving intestinal barrier protection, decreasing intestinal fat transport and modulating intestinal flora.

Combinations as disclosed herein can provide a synergistic treatment in the treatment of NASH:

| TUDCA | TEMPOL |
|---|---|
| Activate bile acid receptors such as farnesoid | |
| X receptor and Takedo G coupled protein | |
| receptor (TGR5) | |
| Inhibits reactive oxygen species (ROS) and | Prevents and reverses the production of |
| inflammation | reactive oxygen species (ROS) and |
| | acts as a SOD mimetic. |
| | Promotes the metabolism of many |
| | reactive oxygen species (ROS) and |
| | improves nitric oxide bioavailability |
| Potent inhibitor of Apoptosis | Positively impacts genes that regulate |
| | cell cycle and programmed cell death |
| | (apoptosis) |
| Interferes with mitochondrial pathway of cell | Restores mitochondrial stability and |
| death | viability |
| Reduces endoplasmic reticulum stress | |
| Stabilizes the unfolded protein response | Significantly decreases expression of |
| | genes encoding for the 26s |
| | proteasome complex, which reduces |
| | the expression of misfolded proteins. |
| Inhibits immune cell activation and | Blocks NF-kB, a key regulator of |
| recruitment | inflammation |
| Reverses fatty liver disease | Can treat and prevent NAFLD in mice |
| | that were fed a high fat diet |
| Improves glucose tolerance, insulin | Has been shown to have a positive |
| secretions, insulin sensitivity, and improves | impact on fatty acid oxidation and plays |
| lipid metabolism | a positive role in glucose and lipid |
| | metabolism |
| Reduces liver fibrosis and protects against | |
| liver cell injury | |
| Inhibits pro-inflammatory cytokines | Downregulates inflammatory cytokines |
| | IL-6, IL-8, IL-1 beta, TNF-alpha, |
| | interferon-gamma, etc. |
| Modulates the intestinal microbiota | |
| composition | |
| Promotes restitution of epithelial cells | Statically increases expression KGF |
| | gene. KGF stimulates proliferation and |
| | differentiation of epithelial cells. |
| | Significantly increases expression of |
| | Glutathione S-Transferase (GST) |
| | gene(s), which is essential for cellular |
| | defense in response to devastating |
| | free radicals. |
| Decreases intestinal fat transport | |
| Improves the intestinal barrier function | |
| Promotes lipid digestion and glycolipid | Positive impact on fatty acid oxidation |
| metabolism | and plays a role in glucose and lipid |
| | metabolism |

Similarly, combination treatments of TUDCA and NAC can be used to treat NASH, in cases with synergistic effects:

| TUDCA | NAC |
|---|---|
| Inhibits reactive oxygen species (ROS) | Acts as an antioxidant and anti- |
| and is anti-inflammatory | inflammatory; scavenging property via the |
| | redox potential of thiols |
| Potent inhibitor of apoptosis | Inhibits apoptosis; reduced expression of |
| | the caspase-3 protein (apoptosis) |
| Interferes with mitochondrial pathway of | Reduces mitochondrial apoptosis and |
| cell death | increases ATP |
| Reduces endoplasmic reticulum (ER) | Administration decreased lipid |
| stress (prevents Grp78 upregulation) | accumulation, apoptosis and ER stress |
| | damage to hepatocytes |
| Modulator of intestinal permeability | Contributes to the tightening of the |
| | intestinal barrier |
| Activates the intestinal farnesoid X | Prevents activity of NFκB which is |
| receptor (FXR); anti-inflammatory effects | involved in the activation of pro- |
| of FXR signaling | inflammatory cytokines |
| Activates TGR5 and leads to decrease in | Decreases multi-pro-inflammatory |
| IL-6, IFNγ and TNFα | cytokines |
| Stabilizes unfolded proteins | Protects proteins by crosslinking cysteine |
| | disulphide molecules. Affects |
| | cytoskeleton structure and restores the |
| | protective effect of unfolded protein |
| | response |
| Inhibits immune activation and recruitment | Produces immuno-modulating activity and |
| | blocks NFκB |
| Activation of bile acid receptors in | Inhibits pro-inflammatory cytokines and |
| immune cells; modulates immune | has immuno-modulating activity |
| response and alters inflammatory | |
| reactions | |
| Inhibits pro-inflammatory cytokines | Downregulates pro-inflammatory |
| | cytokines |
| Improves intestinal barrier function and | Tightens intestinal barrier by influencing |
| intestinal permeability | proteins of the claudin and occludin |
| | groups |
| Modulates intestinal microbiota | Positive effect on intestinal flora |
| composition | |
| Decreases neutrophil infiltration and | Decreases inflammatory neutrophil cell |
| activity as well as a general decrease in | infiltration |
| leukocyte infiltration | |
| Decreases intestinal inflammation | Protects against intestinal inflammation |
| Beneficial effect on systemic and | Has effects on both systemic and |
| intestinal immunity | intestinal immunity |
| Reverses fatty liver disease | Rescues liver steatosis, and ameliorates |
| | intracellular triglyceride accumulation and |
| | preserves mitochondrial function |
| Improves glucose tolerance, insulin | Improves glucose tolerance and protects |
| secretions, insulin sensitivity and | against lipid toxicity |
| improves lipid metabolism | |
| Decreases intestinal fat transport and | Reduces liver fat accumulation |
| promotes lipid digestion | |
| | Significantly restores glutathione |
| | (involved in oxidative metabolism, |
| | inflammatory processes and cellular |
| | regulatory function) |

Treatment of Chemotherapy-Associated Liver Toxicity

Disclosed compositions can be used to prevent, limit, or reduce chemotherapy-associated liver toxicity. Disclosed compositions can be administered prior to, in conjunction with, or following a cancer treatment to prevent, limit, or reduce chemotherapy-associated liver toxicity. For example, in embodiments, disclosed compositions can be administered prior to, concurrently with, or following a chemotherapy regime, such as a chemotherapy regime comprising alkylating agents, antimetabolites, anti-tumor antibiotics, topoisomerase inhibitors, mitotic inhibitors, plant alkaloids, combinations thereof, and the like.

For example, in embodiments, disclosed compositions can be administered prior to, concurrently with, or following a chemotherapy regime comprising any of:

Alkylating agents including:
a. Altretamine
b. Bendamustine
c. Busulfan
d. Carboplatin
e. Carmustine
f. Chlorambucil
g. Cisplatin
h. Cyclophosphamide
i. Dacarbazine
j. Ifosfamide
k. Lomustine
l. Mechlorethamine
m. Melphalan
n. Oxaliplatin
o. Temozolomide
p. Thiotepa
q. Trabectedin

Nitrosoureas including:
a. Carmustine
b. Lomustine
c. Streptozocin

Antimetabolites including:
a. Azacitidine
b. 5-fluorouracil (5-FU)
c. 6-mercaptopurine (6-MP)
d. Capecitabine (Xeloda)
e. Cladribine
f. Clofarabine
g. Cytarabine (Ara-C)
h. Decitabine
i. Floxuridine
j. Fludarabine
k. Gemcitabine (Gemzar)
l. Hydroxyurea
m. Methotrexate
n. Nelarabine
o. Pemetrexed (Alimta)
p. Pentostatin
q. Pralatrexate
r. Thioguanine
s. Trifluidine/tipiracil combination

Anti-tumor antibiotics including:
a. Daunorubicin
b. Doxorubicin (Adriamycin)
c. Doxorubicin liposomal
d. Epirubicin
e. Idarubicin
f. Valrubicin
g. Bleomycin
h. Dactinomycin
i. Mitomycin-C
j. Mitoxantrone (also acts as a topoisomerase II inhibitor)

Topoisomerase I inhibitors (also called camptothecins) including:
a. Irinotecan
b. Irinotecan liposomal
c. Topotecan

Topoisomerase II inhibitors (also called epipodophyllotoxins) including:
a. Etoposide (VP-16)
b. Mitoxantrone (also acts as an anti-tumor antibiotic)
c. Teniposide

Mitotic inhibitors including the taxanes and vinca alkaloids:
a. Taxanes including:
   i. Cabazitaxel
   ii. Docetaxel
   iii. Nab-paclitaxel
   iv. Paclitaxel
b. Vinca alkaloids including:
   i. Vinblastine
   ii. Vincristine
   iii. Vincristine liposomal
   iv. Vinorelbine

Corticosteroids including:
a. Prednisone
b. Methylprednisolone
c. Dexamethasone

As well as other chemotherapy drugs including:
a. All-trans-retinoic acid
b. Arsenic trioxide
c. Asparaginase
d. Eribulin
e. Hydroxyurea
f. Ixabepilone
g. Mitotane
h. Omacetaxine
i. Pegaspargase
j. Procarbazine
k. Romidepsin
l. Vorinostat

Disclosed methods can further comprise radiation therapy.

Thus, disclosed embodiments comprise treatment of chemotherapy-associated liver toxicity with administration of disclosed antioxidant compositions.

Further embodiments can comprise treatments of the conditions in the following Table using disclosed compositions, in some cases with synergistic effects:

| Disease | Therapeutic effect |
|---|---|
| Alzheimer's | Inhibits apoptosis in brain cells to slow |
| | degeneration |
| Parkinson's | Acts as a neuroprotectant |
| Huntington's | Stabilize mitochondrial membrane, |
| | decrease free radical formation, derail |
| | process of cell death |
| Ocular Disorders, e.g. | Reduces retinal cell death and preserves |
| Retinal pigmentosa | function of photoreceptor cells; reduces |
| Macular degeneration | protein misfolding, ER stress, oxidative |
| Retinal detachment | stress, and caspase activity |
| Diabetic retinopathy | |
| Stroke | Cytoprotective in brain injuries; |
| | stabilization of mitochondrial membrane |
| Diabetes | Preserves insulin production by blocking |
| | infiltration of immune cells into the |
| | pancreas; decrease beta cell apoptosis, |
| | preserve insulation secretion, and reduces |
| | ER stress |
| Obesity | Decreases cardiac hypertrophy, high |
| | blood pressure and normalization of |
| | contractility |
| Cardiovascular Disease | Decrease in blood pressure and provide |
| | mitochondrial protection to the heart and |
| | reduction of ER stress |
| Inflammatory Bowel | Protects gastric mucosa from |
| Disease | inflammation; reduces the expression of |
| | inflammatory cytokines, reduces ER |
| | stress and inhibits UPR |
| Acute Kidney Injury | Inhibits mitochondrial pathways of |
| | apoptosis and up-regulates cell survival |
| | pathways and stabilizes the UPR |
| Nonalcoholic | Resolution by histology, improvement in |
| steatohepatitis (NASH) | fibrosis and reduction in progression in |
| | cirrhosis |

Disclosed methods can comprise administration of compositions comprising multiple antioxidants, for example NAC, TEMPOL, and TUDCA. In embodiments comprising two antioxidants, the relative proportion by weight of the two can be, for example, 1:1, 1:2, 1:3, 1:4, 1:5, or the like. Further, the relative proportion by weight of the two can be expressed as A:B, wherein A and B can have values between 1 and 99% and the total percentage is equal to 100.

In embodiments comprising three antioxidants, the relative proportion by weight of the three can be, for example, 1:1:1, 1:2:1, 1:3:1, or the like. Further, the relative proportion by weight of the two can be expressed as A:B:C, wherein A, B, and C can have values between 1 and 99% and the total percentage is equal to 100.

Disclosed compositions can further comprise a pharmaceutically-acceptable carrier.

Disclosed compositions can comprise any pharmaceutically-acceptable form, such as liquids as well as solid dosage forms including pills and tablets. In embodiments, the solid dosage form can dissolve at or above a particular pH value, such as, for example, 7.0.

Disclosed method of treatment can comprise administration of a disclosed composition in an appropriate dosage. For example, in disclosed embodiments, the dosage can comprise daily TUDCA administration of, for example, less than 3000 mg, less than 2900 mg, less than 2800 mg, less than 2700 mg, less than 2600 mg, less than 2500 mg, less than 2400 mg, less than 2300 mg, less than 2200 mg, less than 2100 mg, less than 2000 mg, less than 1900 mg, less than 1800 mg, less than 1700 mg, less than 1600 mg, less than 1500 mg, less than 1400 mg, less than 1300 mg, less than 1200 mg, less than 1100 mg, less than 1000 mg, less than 900 mg, less than 800 mg, less than 700 mg, less than 600 mg, less than 500 mg, less than 400 mg, less than 300 mg, less than 200 mg, less than 100 mg, less than 50 mg, or the like.

In embodiments, the daily dose of TUDCA can comprise between 250 mg and 3000 mg, between 500 mg and 2500 mg, between 1000 mg and 2000 mg, or the like.

In embodiments, the daily dose of TUDCA can comprise 5-35 mg/kg body weight, 10-30 mg/kg body weight, 15-25 mg/kg body weight, or the like.

In embodiments, the daily dose of TUDCA can comprise 25 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, or the like.

Disclosed method of treatment can comprise administration of a disclosed composition in an appropriate dosage. For example, in disclosed embodiments, the dosage can comprise daily TEMPOL administration of, for example, less than 3000 mg, less than 2900 mg, less than 2800 mg, less than 2700 mg, less than 2600 mg, less than 2500 mg, less than 2400 mg, less than 2300 mg, less than 2200 mg, less than 2100 mg, less than 2000 mg, less than 1900 mg, less than 1800 mg, less than 1700 mg, less than 1600 mg, less than 1500 mg, less than 1400 mg, less than 1300 mg, less than 1200 mg, less than 1100 mg, less than 1000 mg, less than 900 mg, less than 800 mg, less than 700 mg, less than 600 mg, less than 500 mg, less than 400 mg, less than 300 mg, less than 200 mg, less than 100 mg, less than 50 mg, or the like.

In embodiments, the daily dose of TEMPOL can comprise between 250 mg and 3000 mg, between 500 mg and 2500 mg, between 1000 mg and 2000 mg, or the like.

In embodiments, the daily dose of TEMPOL can comprise 5-35 mg/kg body weight, 10-30 mg/kg body weight, 15-25 mg/kg body weight, or the like.

In embodiments, the daily dose of TEMPOL can comprise 25 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, or the like.

Disclosed method of treatment can comprise administration of a disclosed composition in an appropriate dosage. For example, in disclosed embodiments, the dosage can comprise daily NAC administration of, for example, less than 3000 mg, less than 2900 mg, less than 2800 mg, less than 2700 mg, less than 2600 mg, less than 2500 mg, less than 2400 mg, less than 2300 mg, less than 2200 mg, less than 2100 mg, less than 2000 mg, less than 1900 mg, less than 1800 mg, less than 1700 mg, less than 1600 mg, less than 1500 mg, less than 1400 mg, less than 1300 mg, less than 1200 mg, less than 1100 mg, less than 1000 mg, less than 900 mg, less than 800 mg, less than 700 mg, less than 600 mg, less than 500 mg, less than 400 mg, less than 300 mg, less than 200 mg, less than 100 mg, less than 50 mg, or the like.

In embodiments, the daily dose of NAC can comprise between 250 mg and 3000 mg, between 500 mg and 2500 mg, between 1000 mg and 2000 mg, or the like.

In embodiments, the daily dose of NAC can comprise 5-35 mg/kg body weight, 10-30 mg/kg body weight, 15-25 mg/kg body weight, or the like.

In embodiments, the daily dose of NAC can comprise 25 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, or the like.

As described above, administration of disclosed embodiments can be via a number of appropriate routes, such as:

| Form | Application | Target |
|---|---|---|
| Liquid | Topical solution for use in | Surface of the eye, particularly in |
| solution | the eye | the back of the eye near the optic |
| | | nerve |
| Tablet | Enters the blood stream via | System wide |
| | the stomach | |
| Capsule | Active agent encapsulated | Surface of the small and large |
| | to delay release until | intestine |
| | reaching the intestine | |
| Sterile | Intravenous injection | System wide |
| solution | | |

Disclosed compositions can be administered in any frequency that results in a therapeutic response. Disclosed methods can comprise use of a loading dose, an initial higher dose of a drug that can be given at the beginning of a course of treatment before dropping down to a lower maintenance dose.

In embodiments, administration can be one, two, three, four, five, or more times a day. In embodiments, administration can be every other day, every third day, every fourth day, every fifth day, every sixth day, once per week, twice per month, monthly, and the like. In an embodiment, administration is once per day.

Commercial Products/Kits

The present compositions and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (e.g. Irgacure 184, 2959), preferably water-soluble initiators (Irgacure 2959). Such irradiation is usually performed for an irradiation time of 1-60 min, but longer irradiation times may be applied, depending on the specific method. Sterile filtration can also be used for sterilization.

The material according to the present disclosure can be finally sterile-wrapped so as to retain sterility until use and packaged (e.g. by the addition of specific product information leaflets) into suitable containers (boxes, etc.).

According to further embodiments, the compositions can also be provided in kit form combined with other components necessary for administration of the material to the patient. For example, combinations may be provided in form of a kit where two or three components are to be administered using different routes of administration, where, for instance, one component is administered using one route of administration and the other component(s) is/are administered using (a) different route(s) of administration.

The kits are designed in various forms based on the specific deficiencies they are designed to treat.

### EXAMPLES

The following non-limiting Examples are provided for illustrative purposes only in order to facilitate a more complete understanding of representative embodiments. This example should not be construed to limit any of the embodiments described in the present Specification.

### Example 1

### Use in CD Treatment

A disclosed composition comprising NAC and TUDCA in a 1:1 w/w ratio is administered to a patient with CD. The solid composition dissolves at a pH value of 7 or greater.

### Example 2

### Use in CD Treatment

A disclosed composition comprising NAC and TEMPOL in a 1:1 w/w ratio is administered to a patient with CD. The solid composition dissolves at a pH value of 7 or greater.

### Example 3

### Use in CD Treatment

A disclosed composition comprising TEMPOL and TUDCA in a 1:2 w/w ratio is administered to a patient with CD. The solid composition dissolves at a pH value of 7 or greater.

### Example 4

### Use in UC Treatment

A disclosed composition comprising NAC and TUDCA in a 1:1 w/w ratio is administered to a patient with UC. The solid composition dissolves at a pH value of 7 or greater.

### Example 5

### Use in UC Treatment

A disclosed composition comprising NAC and TEMPOL in a 1:1 w/w ratio is administered to a patient with UC. The solid composition dissolves at a pH value of 7 or greater.

### Example 6

### Use in UC Treatment

A disclosed composition comprising TEMPOL and TUDCA in a 1:2 w/w ratio is administered to a patient with UC. The solid composition dissolves at a pH value of 7 or greater.

### Example 7

### Use in Macular Degeneration Treatment

A disclosed composition comprising NAC and TUDCA in a 1:1 w/w ratio is administered to a patient with macular degeneration.

### Example 8

### Use in Macular Degeneration Treatment

A disclosed composition comprising NAC and TEMPOL in a 1:1 w/w ratio is administered to a patient with macular degeneration.

### Example 9

### Use in Glaucoma Treatment

A disclosed composition comprising TEMPOL and TUDCA in a 1:2 w/w ratio is administered to a patient with glaucoma.

### Example 10

### Use in Glaucoma Treatment

A disclosed composition comprising NAC and TUDCA in a 1:1 w/w ratio is administered to a patient with glaucoma.

### Example 11

### Use in Glaucoma Treatment

A disclosed composition comprising NAC and TEMPOL in a 1:1 w/w ratio is administered to a patient with glaucoma.

### Example 12

### Use in Glaucoma Treatment

A disclosed composition comprising TEMPOL and TUDCA in a 1:2 w/w ratio is administered to a patient with glaucoma.

### Example 13

### Use in Treatment of FLD

A disclosed composition comprising NAC and TUDCA in a 1:1 w/w ratio is administered to a patient with FLD.

### Example 14

### Use in Treatment of FLD

A disclosed composition comprising NAC and TEMPOL in a 1:1 w/w ratio is administered to a patient with FLD.

### Example 15

### Use in Treatment of FLD

A disclosed composition comprising TEMPOL and TUDCA in a 1:2 w/w ratio is administered to a patient with FLD.

### Example 16

### Use in Treatment of NASH

A disclosed composition comprising NAC and TUDCA in a 1:1 w/w ratio is administered to a patient with NASH.

### Example 17

### Use in Treatment of NASH

A disclosed composition comprising NAC and TEMPOL in a 1:1 w/w ratio is administered to a patient with NASH.

### Example 18

### Use in Treatment of NASH

A disclosed composition comprising TEMPOL and TUDCA in a 1:2 w/w ratio is administered to a patient with NASH.

### Example 19

### Use in Cancer Treatment

A disclosed composition comprising NAC and TUDCA and TEMPOL in a 1:1:1 w/w ratio is administered to a patient prior to chemotherapy.

### Example 20

### Use in Cancer Treatment

A disclosed composition comprising NAC and TUDCA in a 1:1 w/w ratio is administered to a patient following chemotherapy.

### Example 21

### Use in Cancer Treatment

A disclosed composition comprising NAC and TEMPOL in a 1:1 w/w ratio is administered to a patient concurrently with chemotherapy.

### Example 22

### Use in Cancer Treatment

A disclosed composition comprising NAC and TUDCA in a 2:1 w/w ratio is administered to a patient prior to chemotherapy.

### Example 23

### Use in Cancer Treatment

A disclosed composition comprising NAC and TEMPOL in a 2:1 w/w ratio is administered to a patient following chemotherapy.

### Example 24

### Use in Cancer Treatment

A disclosed composition comprising TEMPOL and TUDCA in a 2:1 w/w ratio is administered to a patient concurrently with chemotherapy.

### Example 25

### Use in Cancer Treatment

A disclosed composition comprising NAC and TUDCA in a 1:2 w/w ratio is administered to a patient prior to chemotherapy.

### Example 26

### Use in Cancer Treatment

A disclosed composition comprising NAC and TUDCA in a 1:2 w/w ratio is administered to a patient following chemotherapy.

### Example 27

### Use in Cancer Treatment

A disclosed composition comprising NAC and TUDCA in a 1:2 w/w ratio is administered to a patient concurrently with chemotherapy.

### Example 28

### Use in Glaucoma Treatment

A disclosed composition comprising NAC and TUDCA and TEMPOL in a 1:2:1 w/w ratio is administered to a patient concurrently with glaucoma.

### Example 29

### Use in CD Treatment

Two disclosed liquid compositions comprising NAC and TUDCA (in a 1:1 w/w active ingredient ratio) are administered to a patient with CD.

### Example 30

### Use in CD Treatment

A disclosed solid composition comprising NAC and a liquid composition comprising TEMPOL (in a 1:1 w/w active ingredient ratio) are administered to a patient with CD.

### Example 31

### Use in CD Treatment

A disclosed solid composition comprising TEMPOL and a liquid composition comprising TUDCA (in a 1:2 w/w active ingredient ratio) are administered to a patient with CD.

### Example 32

### Use in UC Treatment

Two disclosed solid compositions (one comprising NAC and one comprising TUDCA) in a 1:1 w/w ratio are administered to a patient with UC.

### Example 33

### Use in UC Treatment

Two disclosed liquid compositions comprising NAC and TEMPOL in a 1:1 w/w ratio are administered to a patient with UC.

### Example 34

### Use in UC Treatment

A disclosed liquid composition comprising TEMPOL and a disclosed solid composition comprising TUDCA (in a 1:2 w/w active ingredient ratio) are administered to a patient with UC.

### Example 35

### Use in Macular Degeneration Treatment

A disclosed liquid composition comprising NAC and TUDCA in a 1:1 w/w ratio is administered to a patient with macular degeneration.

### Example 36

### Use in Macular Degeneration Treatment

A disclosed composition liquid composition comprising NAC and a solid composition comprising TEMPOL (in a 1:1 w/w active ingredient ratio) are administered to a patient with macular degeneration.

### Example 37

### Use in Glaucoma Treatment

A disclosed liquid composition comprising TEMPOL and a solid composition comprising TUDCA (in a 1:2 w/w active ingredient ratio) are administered to a patient with glaucoma.

### Example 38

### Use in Glaucoma Treatment

A disclosed liquid composition comprising NAC and TUDCA (in a 1:1 w/w active ingredient ratio) is administered to a patient with glaucoma.

### Example 39

### Use in Glaucoma Treatment

A disclosed liquid composition comprising NAC and TEMPOL (in a 1:1 w/w active ingredient ratio) is administered to a patient with glaucoma.

### Example 40

### Use in Glaucoma Treatment

A disclosed liquid composition comprising TEMPOL and TUDCA (in a 1:1 w/w active ingredient ratio) is administered to a patient with glaucoma.

### Example 41

### Use in Treatment of FLD

A disclosed powder composition comprising NAC and a disclosed liquid TUDCA (in a 1:1 w/w active ingredient ratio) are administered to a patient with FLD.

### Example 42

### Use in Treatment of FLD

Two disclosed liquid compositions comprising NAC and TEMPOL (in a 1:1 w/w active ingredient ratio) are administered to a patient with FLD.

### Example 43

### Use in CD Treatment

A disclosed composition comprising NAC and TUDCA in a 1:1 w/w ratio is administered to a patient with CD. The solid composition dissolves at a pH value of 7 or greater. The combination of antioxidants produces a synergistic treatment effect.

### Example 44

### Use in CD Treatment

A disclosed composition comprising NAC and TEMPOL in a 1:1 w/w ratio is administered to a patient with CD. The solid composition dissolves at a pH value of 7 or greater. The combination of antioxidants produces a synergistic treatment effect.

### Example 45

### Use in CD Treatment

A disclosed composition comprising TEMPOL and TUDCA in a 1:2 w/w ratio is administered to a patient with CD. The solid composition dissolves at a pH value of 7 or greater. The combination of antioxidants produces a synergistic treatment effect.

### Example 46

### Use in UC Treatment

A disclosed composition comprising NAC and TUDCA in a 1:1 w/w ratio is administered to a patient with UC. The solid composition dissolves at a pH value of 7 or greater. The combination of antioxidants produces a synergistic treatment effect.

### Example 47

### Use in UC Treatment

A disclosed composition comprising NAC and TEMPOL in a 1:1 w/w ratio is administered to a patient with UC. The solid composition dissolves at a pH value of 7 or greater. The combination of antioxidants produces a synergistic treatment effect.

### Example 48

### Use in UC Treatment

A disclosed composition comprising TEMPOL and TUDCA in a 1:2 w/w ratio is administered to a patient with UC. The solid composition dissolves at a pH value of 7 or greater. The combination of antioxidants produces a synergistic treatment effect.

### Example 49

### Use in Macular Degeneration Treatment

A disclosed composition comprising NAC and TUDCA in a 1:1 w/w ratio is administered to a patient with macular degeneration. The combination of antioxidants produces a synergistic treatment effect.

### Example 50

### Use in Macular Degeneration Treatment

A disclosed composition comprising NAC and TEMPOL in a 1:1 w/w ratio is administered to a patient with macular degeneration. The combination of antioxidants produces a synergistic treatment effect.

### Example 51

### Use in Glaucoma Treatment

A disclosed composition comprising TEMPOL and TUDCA in a 1:2 w/w ratio is administered to a patient with glaucoma. The combination of antioxidants produces a synergistic treatment effect.

### Example 52

### Use in Glaucoma Treatment

A disclosed composition comprising NAC and TUDCA in a 1:1 w/w ratio is administered to a patient with glaucoma. The combination of antioxidants produces a synergistic treatment effect.

### Example 53

### Use in Glaucoma Treatment

A disclosed composition comprising NAC and TEMPOL in a 1:1 w/w ratio is administered to a patient with glaucoma. The combination of antioxidants produces a synergistic treatment effect.

### Example 54

### Use in Glaucoma Treatment

A disclosed composition comprising TEMPOL and TUDCA in a 1:2 w/w ratio is administered to a patient with glaucoma. The combination of antioxidants produces a synergistic treatment effect.

### Example 55

### Use in Treatment of FLD

A disclosed composition comprising NAC and TUDCA in a 1:1 w/w ratio is administered to a patient with FLD. The combination of antioxidants produces a synergistic treatment effect.

### Example 56

### Use in Treatment of FLD

A disclosed composition comprising NAC and TEMPOL in a 1:1 w/w ratio is administered to a patient with FLD. The combination of antioxidants produces a synergistic treatment effect.

### Example 57

### Use in Treatment of FLD

A disclosed composition comprising TEMPOL and TUDCA in a 1:2 w/w ratio is administered to a patient with FLD. The combination of antioxidants produces a synergistic treatment effect.

### Example 58

### Use in Treatment of NASH

A disclosed composition comprising NAC and TUDCA in a 1:1 w/w ratio is administered to a patient with NASH. The combination of antioxidants produces a synergistic treatment effect.

### Example 59

### Use in Treatment of NASH

A disclosed composition comprising NAC and TEMPOL in a 1:1 w/w ratio is administered to a patient with NASH. The combination of antioxidants produces a synergistic treatment effect.

### Example 60

### Use in Treatment of NASH

A disclosed composition comprising TEMPOL and TUDCA in a 1:2 w/w ratio is administered to a patient with NASH. The combination of antioxidants produces a synergistic treatment effect.

### Example 61

### Use in Cancer Treatment

A disclosed composition comprising NAC and TUDCA and TEMPOL in a 1:1:1 w/w ratio is administered to a patient prior to chemotherapy. The combination of antioxidants produces a synergistic treatment effect.

### Example 62

### Use in Cancer Treatment

A disclosed composition comprising NAC and TUDCA in a 1:1 w/w ratio is administered to a patient following chemotherapy. The combination of antioxidants produces a synergistic treatment effect.

### Example 63

### Use in Cancer Treatment

A disclosed composition comprising NAC and TEMPOL in a 1:1 w/w ratio is administered to a patient concurrently with chemotherapy. The combination of antioxidants produces a synergistic treatment effect.

### Example 64

### Use in Cancer Treatment

A disclosed composition comprising NAC and TUDCA in a 2:1 w/w ratio is administered to a patient prior to chemotherapy. The combination of antioxidants produces a synergistic treatment effect.

### Example 65

### Use in Cancer Treatment

A disclosed composition comprising NAC and TEMPOL in a 2:1 w/w ratio is administered to a patient following chemotherapy. The combination of antioxidants produces a synergistic treatment effect.

### Example 66

### Use in Cancer Treatment

A disclosed composition comprising TEMPOL and TUDCA in a 2:1 w/w ratio is administered to a patient concurrently with chemotherapy. The combination of antioxidants produces a synergistic treatment effect.

### Example 67

### Use in Cancer Treatment

A disclosed composition comprising NAC and TUDCA in a 1:2 w/w ratio is administered to a patient prior to chemotherapy. The combination of antioxidants produces a synergistic treatment effect.

### Example 68

### Use in Cancer Treatment

A disclosed composition comprising NAC and TUDCA in a 1:2 w/w ratio is administered to a patient following chemotherapy. The combination of antioxidants produces a synergistic treatment effect.

### Example 69

### Use in Cancer Treatment

A disclosed composition comprising NAC and TUDCA in a 1:2 w/w ratio is administered to a patient concurrently with chemotherapy. The combination of antioxidants produces a synergistic treatment effect.

### Example 70

### Use in Glaucoma Treatment

A disclosed composition comprising NAC and TUDCA and TEMPOL in a 1:2:1 w/w ratio is administered to a patient concurrently with glaucoma. The combination of antioxidants produces a synergistic treatment effect.

### Example 71

### Use in CD Treatment

Two disclosed liquid compositions comprising NAC and TUDCA (in a 1:1 w/w active ingredient ratio) are administered to a patient with CD. The combination of antioxidants produces a synergistic treatment effect.

### Example 72

### Use in CD Treatment

A disclosed solid composition comprising NAC and a liquid composition comprising TEMPOL (in a 1:1 w/w active ingredient ratio) are administered to a patient with CD. The combination of antioxidants produces a synergistic treatment effect.

### Example 73

### Use in CD Treatment

A disclosed solid composition comprising TEMPOL and a liquid composition comprising TUDCA (in a 1:2 w/w active ingredient ratio) are administered to a patient with CD. The combination of antioxidants produces a synergistic treatment effect.

### Example 74

### Use in UC Treatment

Two disclosed solid compositions (one comprising NAC and one comprising TUDCA) in a 1:1 w/w ratio are administered to a patient with UC. The combination of antioxidants produces a synergistic treatment effect.

### Example 75

### Use in UC Treatment

Two disclosed liquid compositions comprising NAC and TEMPOL in a 1:1 w/w ratio are administered to a patient with UC. The combination of antioxidants produces a synergistic treatment effect.

### Example 76

### Use in UC Treatment

A disclosed liquid composition comprising TEMPOL and a disclosed solid composition comprising TUDCA (in a 1:2 w/w active ingredient ratio) are administered to a patient with UC. The combination of antioxidants produces a synergistic treatment effect.

### Example 77

### Use in Macular Degeneration Treatment

A disclosed liquid composition comprising NAC and TUDCA in a 1:1 w/w ratio is administered to a patient with macular degeneration. The combination of antioxidants produces a synergistic treatment effect.

### Example 78

### Use in Macular Degeneration Treatment

A disclosed composition liquid composition comprising NAC and a solid composition comprising TEMPOL (in a 1:1 w/w active ingredient ratio) are administered to a patient with macular degeneration. The combination of antioxidants produces a synergistic treatment effect.

### Example 79

### Use in Glaucoma Treatment

A disclosed liquid composition comprising TEMPOL and a solid composition comprising TUDCA (in a 1:2 w/w active ingredient ratio) are administered to a patient with glaucoma. The combination of antioxidants produces a synergistic treatment effect.

### Example 80

### Use in Glaucoma Treatment

A disclosed liquid composition comprising NAC and TUDCA (in a 1:1 w/w active ingredient ratio) is administered to a patient with glaucoma. The combination of antioxidants produces a synergistic treatment effect.

### Example 81

### Use in Glaucoma Treatment

A disclosed liquid composition comprising NAC and TEMPOL (in a 1:1 w/w active ingredient ratio) is administered to a patient with glaucoma. The combination of antioxidants produces a synergistic treatment effect.

### Example 82

### Use in Glaucoma Treatment

A disclosed liquid composition comprising TEMPOL and TUDCA (in a 1:1 w/w active ingredient ratio) is administered to a patient with glaucoma. The combination of antioxidants produces a synergistic treatment effect.

### Example 83

### Use in Treatment of FLD

A disclosed powder composition comprising NAC and a disclosed liquid TUDCA (in a 1:1 w/w active ingredient ratio) are administered to a patient with FLD. The combination of antioxidants produces a synergistic treatment effect.

### Example 84

### Use in Treatment of FLD

Two disclosed liquid compositions comprising NAC and TEMPOL (in a 1:1 w/w active ingredient ratio) are administered to a patient with FLD. The combination of antioxidants produces a synergistic treatment effect.

In closing, it is to be understood that although aspects of the present Specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present Specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the Specification is deemed to contain the group as modified thus fulfilling the written description of all Markush groups used in the appended claims.

Unless otherwise indicated, all numbers expressing a characteristic, item, quantity, parameter, property, term, and so forth used in the present Specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the Specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present Specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present Specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

What is claimed is:

1. A method for treating inflammatory bowel disease (IBD) comprising administering at least two antioxidants to a patient in need thereof.

2. The method of claim 1, wherein said IBD comprises Crohn's Disease (CD).

3. The method of claim 1, wherein said IBD comprises Ulcerative Colitis (UC).

4. A method for treating glaucoma comprising administering at least two antioxidants to a patient in need thereof.

5. A method for treating macular degeneration comprising administering at least two antioxidants to a patient in need thereof.

6.-12. (canceled)

13. The method of claim 1, wherein said administering comprises daily administration.

14. The method of claim 1, wherein said administering comprises weekly administration.

15. The method of claim 1, wherein said administering comprises monthly administration.

16. The method of claim 1 wherein one of said at least two antioxidants comprises NAC.

17. The method of claim 1, wherein one of said at least two antioxidants comprises TUDCA.

18. The method of claim 1, wherein one of said at least two antioxidants comprises TEMPOL.

19. The method of claim 1, wherein said at least two antioxidants comprise a single composition.

20. The method of claim 19, wherein said single composition comprises a solid composition.

21. The method of claim 19, wherein said single composition comprises a liquid composition.

22.-48. (canceled)

49. A composition comprising at least two antioxidants.

50. The composition of claim 49, wherein one of said at least two antioxidants comprises NAC.

51. The composition of claim 50, wherein one of said at least two antioxidants comprises TUDCA.

52. The composition claim 50, wherein one of said at least two antioxidants comprises TEMPOL.

53. The composition of claim 50, wherein said composition is a solid.

54. The composition of claim 50, wherein said composition is a liquid.

55. (canceled)
